(19) United States
(12) Patent Application Publication
(10) Pub. No.: US 2025/0223203 A1
(43) Pub. Date: Jul. 10, 2025

(54) INTELLIGENT DOSING CONTROL METHOD, DEVICE AND SYSTEM FOR SOFTENING AND REMOVING HARDNESS

(71) Applicant: BEIJING XINYUAN SMART WATER GROUP CO., LTD, Beijing (CN)

(72) Inventors: Hong WANG, Beijing (CN); Hong LIU, Beijing (CN); Jiangang HUI, Beijing (CN)

(73) Assignee: BEIJING XINYUAN SMART WATER GROUP CO., LTD, Beijing (CN)

(21) Appl. No.: 19/001,512

(22) Filed: Dec. 25, 2024

(30) Foreign Application Priority Data
Jan. 4, 2024 (CN) 202410013716.9
Jan. 11, 2024 (CN) 202410043580.6

(51) Int. Cl. C02F 1/52; C02F 5/06

(52) U.S. Cl. CPC C02F 1/5209; C02F 1/5245; C02F 5/06; C02F 2209/001; C02F 2209/02; C02F 2209/055; C02F 2209/06; C02F 2209/07; C02F 2209/40; C02F 2209/44

(57) ABSTRACT

The disclosure relates to an intelligent dosing control method, device and system for softening and removing hardness. Based on calculation by the intelligent dosing control method or device for softening and removing hardness of the disclosure, a softening agent dosing scheme can be intelligently recommended, while allowing the dosage adjustment in real time, thereby realizing the real-time and precise dosing of agents throughout the process. The intelligent dosing control system for softening and removing hardness of the disclosure automatic monitors the inlet water quality, and the dosing device can be controlled to perform precise dosing of agents through the calculation by the intelligent dosing control method or device for softening and removing hardness. Based on the monitoring feedback of outlet water quality and then calculation by the intelligent dosing control method and/or device for softening and removing hardness, the agent dosage can be automatically readjusted.

### CROSS-REFERENCE TO RELATED APPLICATION

This application claims the priority benefit of China application serial no. 202410013716.9, filed on Jan. 4, 2024 and China application serial no. 202410043580.6, filed on Jan. 11, 2024. The entirety of each of the above-mentioned patent applications is hereby incorporated by reference herein and made a part of this specification.

### BACKGROUND

### Technical Field

This disclosure relates to an intelligent dosing control method, device and system for softening and removing hardness, which belongs to the field of hardness treatment of industrial wastewater.

### Description of Related Art

Industrial wastewater contains a large amount of scaling ions: calcium, magnesium, and bicarbonate. In the process of industrial wastewater reuse treatment or zero-discharge treatment to produce qualified salts, the hardness removal effect is often unsatisfactory, which often leads to membrane fouling and substandard salt quality. The fluctuation of water quality, the lag of water quality laboratory analysis and the inconsistent levels of operators, all make it difficult to ensure the hardness removal effect and outlet water quality, and even dosing mistaken agents or an overdose will result in a waste of operating costs. At present, the agent dosing control method mainly involves using the inlet water quality feed forward and water quality feedback for PID regulation. The fluctuation of water quantity and quality may lead to difficulties in PID regulation, even making it challenging to converge the agent dosage and to meet the expected water quality. Therefore, the precise dosage of softening agents in the softening and hardness removal process section is the key and difficult point in operation.

A patent with the application No. 201911324558.4 discloses a BP neural network-based intelligent dosing control system, comprising an inlet water flow meter for acquiring an inlet water volume value; an inlet water total phosphorus detector for collecting a total phosphorus value of inlet water; an ATV phosphorus removal improvement model for introducing the inlet water volume value, the total phosphorus value of inlet water for real-time calculation to obtain the dosing trend value and the dosage; an outlet water total phosphorus detector for acquiring a total phosphorus value of outlet water; a BP neural network adjustment module for introducing the received dosage and the total phosphorus value of outlet water to calculate the dosage correction value; a PLC control module for converting the dosage correction value into a corresponding control signal; a dosing pump for dosing according to the control signal. By acquiring the inlet water volume value, the total phosphorus value of inlet water for real-time calculation of the dosing trend value and dosage, introducing the dosage and the total phosphorus value of outlet water for calculation of the dosage correction value, and dosing agents into the clarifier based on the dosage correction value of the control system, the phosphorus removal efficiency of the clarifier has been improved.

However, the system aims to wastewater with phosphorus to be removed, this wastewater composition is relatively simple, and the agent dosing of the dosing control system is simple, it is impossible to perform accurate dosing treatment for wastewater with different compositions and contents of impurities.

### SUMMARY

In view of the defects of the prior art, the present disclosure provides an intelligent dosing control method, device and system for softening and removing hardness, which automatically and intelligently recommends a softening agent dosing scheme according to the impurity composition of industrial wastewater, thereby solving the problem of low degree of automation in the process of removing hardness of industrial wastewater, realizing real-time and precise dosing throughout the process, ensuring the hardness removal effect of the system, as well as avoiding adverse losses caused by operators due to differences in quality and human operation.

In order to achieve the above objects, the present disclosure proposes the following technical solutions.

In a first aspect, an intelligent dosing control method for softening and removing hardness is provided, comprising:
(1) recommending an agent scheme, including: analyzing and comparing a plurality of agent dosing schemes based on different inlet water qualities and produced water quality requirements, and then recommending an appropriate agent dosing scheme based on the agent cost and sludge production;
(2) precisely controlling the agent dosing, including:
a first level: accurate data calculation, wherein a theoretical agent dosage is obtained by calculating accurately and analyzing the inlet water flow rate and the calcium ion, magnesium ion, and alkalinity in the inlet and outlet water, yₘ represents the theoretical agent dosage obtained after the m-th accurate calculation and analysis, where m is a positive integer greater than or equal to 1;
a second level: fuzzy data adjustment, comprising a first stage of memory and analysis and a second stage of output and readjustment;
in the first stage of memory and analysis: dosing by taking yₘ (m≤n) obtained from accurate data calculation of the first level as the basis for the expected agent dosing, then performing analysis and adjustment of the agent dosage and a secondary dosing according to the feedback of the produced water quality, and recycling the analysis and adjustment of agent dosage based on the feedback of produced water quality until the produced water quality meets a requirement, stopping the recycle, performing statistics of the actual agent consumption to obtain an actual agent dosage y; then conducting fuzzy factor function analysis y=f(yₘ, X) based on y and yₘ to give the fuzzy factor function f(yₘ, X), where X represents influencing factors that have no determined impact on the outlet water quality, the fuzzy factor function f(yₘ, X) is then combined with the actual inlet and outlet water quality as well as the agent dosing scheme and the agent dosing parameters yₘ and y to form a multi-dimensional data point, which is stored in memory; upon a memory database is formed by accumulating n multi-dimensional data points, entering the second stage of output and readjustment, where n is a positive integer greater than or equal to 1000;
in the second stage of output and readjustment: simultaneously introducing the inlet water and produced water quality requirements and yₘ (m>n) obtained according to accurate data calculation of the first level into the fuzzy data adjustment of the second level, and performing comparison and analysis with the multi-dimensional data points in the memory database to give the corresponding fuzzy factor function f(yₘ, X), and outputting an agent dosing parameter including y corresponding to f(yₘ, X), then performing a predictable agent dosing according to the parameter using y as the predictable dosing basis; after receiving the feedback of actual produced water quality, performing comparison and analysis with the produced water quality requirements: if there is no difference, determining that the predictable agent parameter adjustment and dosing are achievable; if there is a difference, performing readjustment of the agent dosing according to the actual produced water quality and requirements on produced water quality, i.e., recycling the analysis and adjustment of agent dosage based on the feedback of produced water quality until the actual produced water quality meets the requirement on the produced water quality, stopping the recycle, performing statistics of the actual agent consumption to obtain an actual agent dosage z; then conducting fuzzy factor function analysis z=f(y, X) based on y and z to give the fuzzy factor function f(y, X), thereby realizing the readjustment of the fuzzy factor function f(yₘ, X), while forming a new multi-dimensional data point including the actual inlet and outlet water quality, the agent dosing scheme, y, z, and f(y, X), which is stored in memory; thereby realizing the intelligent agent dosing control through continuous output adjustment and memory.

Further, there are at least two agent dosing schemes.

Further, there are at most seven agent dosing schemes.

Still further, the agent dosing scheme includes two or more of model 1: calcium hydroxide; model 2: calcium hydroxide+sodium carbonate; model 3: calcium hydroxide+sodium carbonate+trisodium phosphate; model 4: sodium hydroxide; model 5: calcium hydroxide+sodium hydroxide; model 6: sodium hydroxide+sodium carbonate; model 7: lime+gypsum or lime+calcium chloride.

Still further, the model 1 is suitable for wastewater with low hardness and high alkalinity; and/or
the model 2 is suitable for wastewater with high hardness and low alkalinity; and/or
the model 3 is suitable for wastewater which is treated with lime+soda ash to give the residual hardness of 0.15-0.2 mmol/L, and needs further softening to obtain a residual hardness of 10-20 μmol/L; and/or
the model 4 is suitable for wastewater where the 2× carbonate alkalinity of raw water is not less than the calcium hardness of raw water; preferably, the model 4 is suitable for wastewater where the 2× carbonate alkalinity of raw water is equal to the calcium hardness of raw water; the model 5 is suitable for wastewater where the 2× carbonate alkalinity of raw water is more than the calcium hardness of raw water; and/or
the model 6 is suitable for wastewater where the 2× carbonate alkalinity of raw water is less than the calcium hardness of raw water; and/or
the model 7 is suitable for negative hard water having a total alkalinity of raw water greater than or equal to a total hardness of raw water of 2 mmo/L.

Further, the influencing factor X includes one or more of the accuracy of various measuring instruments, the fluid discharge condition of the dosing device, the operating temperature and the reaction time.

In a second aspect, an intelligent dosing control device for softening and removing hardness is provided. The intelligent dosing control device for softening and removing hardness employs the above intelligent dosing control method for softening and removing hardness.

In a third aspect, an intelligent dosing control system for softening and removing hardness is provided, which comprises the above intelligent dosing control device for softening and removing hardness.

Further, the intelligent dosing control system for softening and removing hardness further comprises monitoring instruments, arranged at the inlet pipe of the softening and hardness removal process section, for monitoring flow rate, hardness, Ca ion-like ions, alkalinity, pH and conductivity, to transmit the inlet water monitoring data to the intelligent dosing control device for softening and removing hardness; and
an agent dosing device and a hardness removal device, wherein the intelligent dosing control device for softening and removing hardness outputs an agent dosage to the agent dosing device, to enable the agent to be accurately dosed into the hardness removal device for softening and removing hardness of the wastewater; and
monitoring instruments, arranged at the outlet pipe of the hardness removal device, for monitoring flow rate, hardness, Ca ion-like ions, alkalinity, pH and conductivity, to transmit the outlet water monitoring data to the intelligent dosing control device for softening and removing hardness.

Still further, the monitoring instruments are online real-time monitoring instruments that measure inlet and/or outlet water in real time; preferably, the frequency of monitoring statistics data is once every 2 hours.

Compared with the prior art, the present disclosure has the following beneficial effects:

1. Based on calculation by the intelligent dosing control method for softening and removing hardness of the disclosure, a softening agent dosing scheme can be intelligently recommended, while allowing the dosage adjustment in real time, thereby realizing the real-time and precise dosing of agents throughout the process.

2. The intelligent dosing control system for softening and removing hardness of the disclosure enables automatic monitoring of the inlet water quality, and the dosing device can be controlled to enable precise dosing of agents through the calculation by the intelligent dosing control method or device for softening and removing hardness. Based on the monitoring feedback of outlet water quality and then calculation by the intelligent dosing control method and/or device for softening and removing hardness, the agent dosage can be automatically readjusted.

3. The effect of removing wastewater hardness can be guaranteed by the intelligent dosing control method, device and system for softening and removing hardness of the disclosure, while avoiding adverse losses caused by operators due to differences in quality and human operation.

### BRIEF DESCRIPTION OF THE DRAWINGS

FIG. 1 shows a schematic diagram of the internal logic of the intelligent dosing control method of the present disclosure;

FIG. 2 shows a schematic diagram of the structure of an intelligent dosing control system for softening and removing hardness according to an embodiment of the present disclosure.

### DESCRIPTION OF THE EMBODIMENTS

In order to make the objects, technical solutions and advantages of the disclosure clearer, the disclosure will be described clearly and completely with specific embodiments below. The embodiments of the disclosure are implemented on the premise of the technical solution of the disclosure, and detailed implementations and processes are set forth. However, the scope of protection of the disclosure is not limited to the following embodiments. Those skilled in the art should understand that the embodiments are only to assist in the understanding of the present disclosure, and should not be regarded as specific limitations of the disclosure. Based on the embodiments of the present disclosure, all other embodiments obtained by those of ordinary skill in the art without creative work are within the scope of the present disclosure.

Neither the endpoints nor any of the values in the ranges disclosed in the present disclosure are limited to that precise range or value, and those ranges or values should be understood to include values close to those ranges or values. For numerical ranges, one or more new numerical ranges may be obtained by combining between the endpoint values of the ranges, between the endpoint values of the ranges and individual point values, and between individual point values, which shall be deemed to be specifically disclosed in the present disclosure.

Unless otherwise specified and/or indicated, all values relating to the amount of a component are “by weight” herein. The process parameters with no specific conditions indicated in the following embodiments usually follow the conventional conditions. The raw materials described in the following embodiments are all commercially available from open commercial sources.

Specific implementations of the present disclosure are described in detail below. It will be understood that the specific implementations described herein are only used to illustrate and explain the disclosure, and are not intended to limit the disclosure.

According to the first aspect of the present disclosure, provided is an intelligent dosing control method for softening and removing hardness, comprising:
(1) recommending an agent scheme, including: analyzing and comparing a plurality of agent dosing schemes based on different inlet water qualities and produced water quality requirements, and then recommending an appropriate agent dosing scheme based on the agent cost and sludge production;
(2) precisely controlling the agent dosing, including:
a first level: accurate data calculation, wherein a theoretical agent dosage is obtained by calculating accurately and analyzing the inlet water flow rate and the calcium ion, magnesium ion, and alkalinity in the inlet and outlet water, yₘ represents the theoretical agent dosage obtained after the m-th accurate calculation and analysis, where m is a positive integer greater than or equal to 1;
a second level: fuzzy data adjustment, comprising a first stage of memory and analysis and a second stage of output and readjustment;
in the first stage of memory and analysis: dosing by taking yₘ (m≤n) obtained from accurate data calculation of the first level as the basis for the expected agent dosing, then performing analysis and adjustment of the agent dosage and a secondary dosing according to the feedback of the produced water quality, and recycling the analysis and adjustment of agent dosage based on the feedback of produced water quality until the produced water quality meets a requirement, stopping the recycle, performing statistics of the actual agent consumption to obtain an actual agent dosage y; then conducting fuzzy factor function analysis y=f(yₘ, X) based on y and yₘ to give the fuzzy factor function f(yₘ, X), where X represents influencing factors that have no determined impact on the outlet water quality, the fuzzy factor function f(yₘ, X) is then combined with the actual inlet and outlet water quality as well as the agent dosing scheme and the agent dosing parameters yₘ and y to form a multi-dimensional data point, which is stored in memory; upon a memory database is formed by accumulating n multi-dimensional data points, entering the second stage of output and readjustment, where n is a positive integer greater than or equal to 1000;
in the second stage of output and readjustment: simultaneously introducing the inlet water and produced water quality requirements and yₘ (m>n) obtained according to accurate data calculation of the first level into the fuzzy data adjustment of the second level, and performing comparison and analysis with the multi-dimensional data points in the memory database to give the corresponding fuzzy factor function f(yₘ, X), and outputting an agent dosing parameter including y corresponding to f(yₘ, X), then performing a predictable agent dosing according to the parameter using y as the predictable dosing basis; after receiving the feedback of actual produced water quality, performing comparison and analysis with the produced water quality requirements: if there is no difference, determining that the predictable agent parameter adjustment and dosing are achievable; if there is a difference, performing readjustment of the agent dosing according to the actual produced water quality and requirements on produced water quality, i.e., recycling the analysis and adjustment of agent dosage based on the feedback of produced water quality until the actual produced water quality meets the requirement on the produced water quality, stopping the recycle, performing statistics of the actual agent consumption to obtain an actual agent dosage z; then conducting fuzzy factor function analysis z=f(y, X) based on y and z to give the fuzzy factor function f(y, X), thereby realizing the readjustment of the fuzzy factor function f(yₘ, X), while forming a new multi-dimensional data point including the actual inlet and outlet water quality, the agent dosing scheme, y, z, and f(y, X), which is stored in memory; thereby realizing the intelligent agent dosing control through continuous output adjustment and memory.

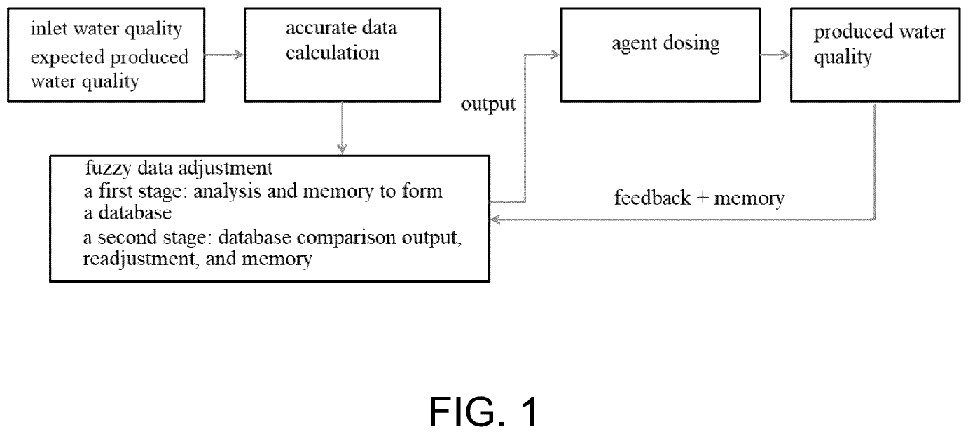

The intelligent dosing control method for softening and removing hardness (intelligent dosing control method) of the disclosure mainly realizes two functions: (1) agent scheme recommendation, and (2) accurate agent dosing control, the internal logic schematic diagram of which is shown in FIG. 1. Firstly, (1) agent scheme recommendation: in order to meet different requirement of inlet water qualities and produced water quality, a variety of built-in softening agent dosing schemes are established to perform analysis and comparison of various softening agent dosing schemes according to the water quality, and then an appropriate agent dosing scheme may be recommended based on the agent cost and sludge production.

Commonly used agents for softening hard water are lime, sodium carbonate, sodium hydroxide, trisodium phosphate, disodium hydrogen phosphate, etc. According to the raw water quality and process requirements, one or several agents may be used simultaneously, different agents are suitable for different water quality. Therefore, according to the inlet water quality, one or more built-in agent dosing schemes are comprised. At least two agent dosing schemes are set to enable comparison and analysis of agent schemes to make a recommendation. At most seven agent dosing schemes are set to meet the hardness removal needs of most wastewater.

Further, the agent dosing scheme includes two or more of model 1: calcium hydroxide; model 2: calcium hydroxide+sodium carbonate; model 3: calcium hydroxide+sodium carbonate+trisodium phosphate; model 4: sodium hydroxide; model 5: calcium hydroxide+sodium hydroxide; model 6: sodium hydroxide+sodium carbonate; model 7: lime+gypsum or lime+calcium chloride. Among them, model 1: calcium hydroxide is suitable for wastewater with low hardness and high alkalinity; model 2: calcium hydroxide+sodium carbonate is suitable for wastewater with high hardness and low alkalinity; model 3: calcium hydroxide+sodium carbonate+trisodium phosphate is suitable for wastewater which is treated with lime+soda ash (Na₂CO₃) or model 2 to give the residual hardness of 0.15-0.2 mmol/L, and needs further softening to obtain a residual hardness of 10-20 μ mol/L; model 4 is suitable for wastewater where the 2× carbonate alkalinity of raw water is not less than the calcium hardness of raw water; preferably, model 4 is suitable for wastewater where the 2× carbonate alkalinity of raw water is equal to the calcium hardness of raw water; model 5: calcium hydroxide+sodium hydroxide is suitable for wastewater where the 2× carbonate alkalinity of raw water is more than the calcium hardness of raw water; model 6: sodium hydroxide+sodium carbonate is suitable for wastewater where the 2× carbonate alkalinity of raw water is less than the calcium hardness of raw water; model 7: lime+gypsum (CaSO₄·2H₂O) or lime+calcium chloride is suitable for negative hard water having a total alkalinity of raw water greater than or equal to a total hardness of raw water of 2 mmo/L.

### (2) Accurate Agent Dosing Control:

The intelligent dosing control analysis comprises two levels: accurate data calculation and fuzzy data adjustment.

A first level: accurate data calculation, specifically, a theoretical agent dosage is obtained by calculating accurately and analyzing the water flow rate, calcium ion, magnesium ion, and alkalinity of the inlet water, and the calcium ion, magnesium ion, and alkalinity required in the outlet water, wherein ym represents the theoretical agent dosage obtained after the m-th accurate calculation, where m is a positive integer greater than or equal to 1. When performing accurate data calculation, since the agent softening process used in removing hardness of wastewater is based on the chemical precipitation principle, which enables scaling ions in water to be removed by forming insoluble compounds according to the solubility product principle, and needs to be carried out combined with the coagulation and precipitation process. Thus, the process needs to be carried out according to a specific chemical reaction and precipitation solubility product based on the wastewater and the agent dosing scheme. Accurate data calculation is illustrated by example below. Preferably, the specific agent softening treatment way and applicable water quality are shown in the following table:

| Model | Agent dosing scheme | Applicable water quality |
|---|---|---|
| 1 | Calcium hydroxide | A₀ > H₀ |
| 2 | Calcium hydroxide + | H₀ > A₀ |
| | sodium carbonate | |
| 3 | Calcium hydroxide + | water having a residual hardness of |
| | sodium carbonate + | 0.15-0.2 mmol/L after being treated |
| | trisodium phosphate | with lime + soda ash (model 2), |
| | | which needs further softening to |
| | | obtain a residual hardness of |
| | | 10-20 μmol/L |
| 4 | Sodium hydroxide | 2H_{z} + CO₂ ≥ (preferably=)H_{Ca} + |
| | | K + β |
| 5 | Calcium hydroxide + | 2H_{z} + CO₂ > H_{Ca} + K + β |
| | sodium hydroxide | |
| 6 | Sodium hydroxide + | 2H_{z} + CO₂ < H_{Ca} + K + β |
| | sodium carbonate | |
| 7 | Lime + gypsum or | A₀ − H₀ > 2 mmol/L negative |
| | lime + calcium | hard water |
| | chloride | |

In the table:
H_{Ca}: calcium hardness in raw water (mmol/L);
A₀: total alkalinity in raw water (mmol/L);
H₀: total hardness in raw water (mmol/L);
H_{z}: carbonate hardness in raw water (mmol/L);
K: flocculant dosage (mmol/L), generally 0.05-0.25 mmol/L;
β: overdosage of CO₃²⁻ (mmol/L), generally 0.5-0.7 mmol/L;
Lime: CaO; soda ash: Na₂CO₃; gypsum: CaSO₄·2H₂O;

Calcium hydroxide can also be replaced by calcium oxide, which, when dissolved in water, reacts to form calcium hydroxide. Considering the influence of impurities contained in calcium oxide, calcium hydroxide is preferred; it is also possible to dissolve lime in water to from a calcium hydroxide solution with a certain concentration, which is used after removing some impurities to reduce the cost, wherein the concentration of the calcium hydroxide solution is set according to the needs, for example, as 5%. Furthermore, model 4 is suitable for wastewater where the 2× carbonate alkalinity of raw water is greater than or equal to the calcium hardness of raw water, preferably for wastewater where the 2× carbonate alkalinity of raw water is equal to the calcium hardness of raw water. When the 2× carbonate alkalinity of raw water is greater than the calcium hardness of raw water, calcium hydroxide is preferably used to replace parts of calcium hydroxide in model 4, that is, forming a new agent dosing model, model 5, thereby ensuring the water treatment effect while reducing the cost.

Depending on the water quality and treatment requirements, the most commonly used agent softening models are: model 1, model 2, and model 6. The chemical reactions that take place in the three softening treatment models are described below:

### Softening Treatment of Model 1:

Model 1: calcium hydroxide, i.e., a lime softening method, is suitable for water with low hardness and high alkalinity to remove temporary hardness from the water, with the following reactions:

Ca(HCO₃)₂+Ca(OH)₂→2CaCO₃↓+2H₂O

Mg(HCO₃)₂+Ca(OH)₂→MgCO₃↓+CaCO₃↓+2H₂O

MgCO₃+Ca(OH)₂→Mg(OH)₂↓+CaCO₃↓

### Softening Treatment of Model 2:

Model 2: lime+sodium carbonate, is suitable for water with high hardness and low alkalinity. Lime is used to remove carbonate hardness from the water (with the reaction equation shown in softening treatment of model 1) and sodium carbonate is used to remove non-carbonate hardness from the water, with the following reaction equations:

CaSO₄+Na₂CO₃→CaCO₃↓+Na₂SO₄

CaCl₂+Na₂CO₃→CaCO₃↓+2NaCl

MgSO₄+Na₂CO₃→MgCO₃↓+Na₂SO₄

MgCl₂+Na₂CO₃→MgCO₃↓+2NaCl

MgCO₃ hydrolyzes quickly at higher pH:

MgCO₃+H₂O→Mg(OH)₂↓+CO₂↓

Softening treatment of model 6: Sodium hydroxide+sodium carbonate, is suitable for water with 2 times the carbonate alkalinity less than the calcium hardness, with the following reactions:

Ca(HCO₃)₂+2NaOH→2CaCO₃↓+Na₂CO₃+2H₂O

Mg(HCO₃)₂+4NaOH→Mg(OH)₂↓+2Na₂CO₃+2H₂O

CO₂+2NaOH→Na₂CO₃+H₂O

CaSO₄+Na₂CO₃→CaCO₃↓+Na₂SO₄

CaCl₂+Na₂CO₃→CaCO₃↓+2NaCl

MgSO₄+2NaOH→Mg(OH)₂↓+Na₂SO₄

MgCl₂+2NaOH→Mg(OH)₂↓+2NaCl

A second level: fuzzy data adjustment. Usually when dosing with an expected agent dosage, the outlet water quality cannot meet the expected requirements due to a variety of factors in practice. Thus, there is a need for correction of the expected agent dosage according to the actual situation to meet the expected requirements. In practice, the accuracy of the measuring instrument, the discharge of the dosing pump, the reaction temperature, the reaction time and so on will affect the outlet water quality. Specifically, for example, the dosing pump uses a mechanical diaphragm metering pump, which normally has a 2% error of pump delivering flow rate, and if employing a screw pump, there also will be a certain flow error. For another example, when the agent reacts with wastewater, the temperature will affect the speed of the reaction, which in turn has an effect on the outlet water quality. For another example, an appropriate reaction time will be considered when designing the water flow rate, such as designing the reaction time as 2 min. When water fluctuation is significant in practice, the designed reaction time may be insufficient, which in turn affects the outlet water quality. Equation 1 below shows: the effects of various influencing factors X on the actual agent dosage y and the expected agent dosage y₀.

$$y = f ( {y}_{0} , X ) \qquad Equation 1$$

These influencing factors do not have a determined influencing relationship with the outlet water quality, and even some factors cannot get the measured data due to the difficulty in monitoring in field. For example, the error values of instruments per se, i.e., the error values that still exist after calibration of the flow meters, which will become greater when the operating conditions differ from the calibration conditions significantly. For another example, the purity of agents, i.e., the influence of impurities in agents. Therefore, the actual influencing factors X together with the fuzzy factor function f are used as the basis for fuzzy adjustment to correct, memorize and output the agent dosage.

In particular, the second level of fuzzy data adjustment is divided into two stages: a first stage of memory and analysis, and a second stage of output and readjustment.

The first stage of memory and analysis: by taking the agent dosage y₁ obtained from accurate data calculation of the first level for the first time as the basis for the expected agent dosing, the agent dosing device are controlled for dosing; then analysis and adjustment are performed according to the feedback of the hardness removal effect of produced water, after the adjustment of the agent dosage, the agent dosing device are controlled for dosing until the produced water quality reaches the expected effect; statistics of the actual dosage is performed to give the adjusted dosage, i.e., the actual agent dosage y; and then by taking the actual influencing factors X as the basis for fuzzy adjustment, a fuzzy factor function analysis y=f(y₁, X) is performed based on y and y₁ to obtain the fuzzy factor function f(y₁, X), which is combined with the inlet and outlet water quality at this point (such as the inlet water flow rate, inlet water pH, outlet water pH, calcium ion concentration/magnesium ion concentration/alkalinity concentration of inlet water, calcium ion concentration/magnesium ion concentration/alkalinity of outlet water), control parameters for the agent dosing device (i.e., agent dosing parameters, such as the agent dosing scheme, dosing pump operating frequency, agent dosing flow rate including y₁ and y), and so on, to form a multi-dimensional data point, which is stored in memory. After continued operation for a period of time (such as 3 months) on the pretreatment water quality by the intelligent dosing control method, n multi-dimensional data points can be accumulated (n≥1000, preferably at least 12 data points can be formed within 24 hours, if the inlet water quality and flow rate are extremely unstable, the operation time and the data point quantity n need to be increased) to form a memory database. At this point, the second stage of output and readjustment is entered.

The second stage of output and readjustment: simultaneously introducing yₘ (m>n) obtained according to accurate data calculation of the first level of the n+1-th and subsequent times and the inlet water quality of this time and produced water quality requirements into the fuzzy data adjustment of the second level and performing comparison and analysis with the multi-dimensional data point in the memory database to give the fuzzy factor function f(yₘ, X), simultaneously outputting an agent dosing parameter including y corresponding to f(yₘ, X), and then performing a predictable agent dosing based on y. After the feedback of actual produced water quality is received, it may be compared and analyzed with the expected produced water quality requirements: if there is no difference, determining that the predictable agent parameter adjustment and efficient control of the agent dosing device for dosing are achievable; if there is a difference, performing readjustment of the agent dosing according to the actual produced water quality and requirements on produced water quality, i.e., recycling the analysis and adjustment of agent dosage based on the feedback of produced water quality, until the actual produced water quality meets the requirements on the produced water quality, then stopping the recycle, and performing statistics of the actual agent dosage to obtain an actual agent dosage z, thereby realizing the readjustment of the agent dosage data. Then fuzzy factor function analysis z=f(y, X) is performed based on y and z to give the fuzzy factor function f(y, X), thereby realizing the readjustment of the fuzzy factor function f(yₘ, X), while forming a new multi-dimensional data point including the actual inlet and outlet water qualities, the agent dosing scheme, y, z, and f(y, X). As such, the intelligent control of agent dosing is realized through continuous output, adjustment and memory.

According to a second aspect of the disclosure, an intelligent dosing control device for softening and removing hardness is provided, which employs the intelligent dosing control method for softening and removing hardness described above.

According to a third aspect of the disclosure, an intelligent dosing control system for softening and removing hardness is provided, which comprises the intelligent dosing control device for softening and removing hardness described above.

As an alternative implementation of the intelligent dosing control system for softening and removing hardness, the intelligent dosing control system for softening and removing hardness further comprises: monitoring instruments, arranged at the inlet pipe of the softening and hardness removal process section, for monitoring flow rate, hardness, calcium ions and similar ions, alkalinity, pH and conductivity, to transmit the inlet water monitoring data to the intelligent dosing control system for softening and removing hardness; and
an agent dosing device and a hardness removal device, wherein the intelligent dosing control device for softening and removing hardness outputs an agent dosage to the agent dosing device, to enable the agent to be accurately dosed into the hardness removal device for softening and removing hardness of wastewater; and
monitoring instruments, arranged at the outlet pipe of the hardness removal device, for monitoring flow rate, hardness, Ca ion-like ions, alkalinity, pH and conductivity, to transmit the outlet water monitoring data to the intelligent dosing control device for softening and removing hardness.

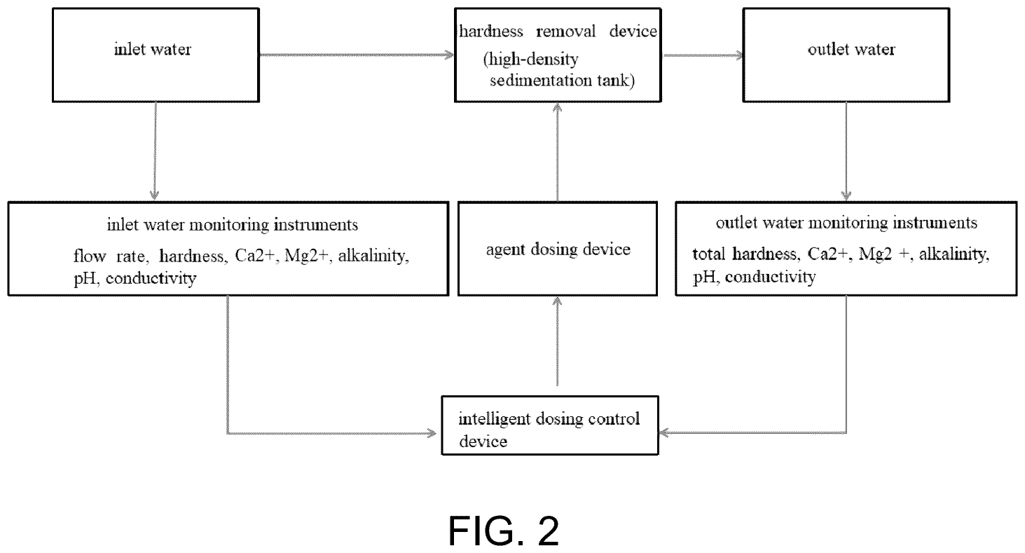

In the above technical solution, a schematic diagram of the structure of the intelligent dosing control system for softening and removing hardness is shown in FIG. 2:
monitoring instruments are arranged at the inlet pipe of the softening and hardness removal process section, for monitoring flow rate, hardness, calcium ions and similar ions, alkalinity, pH and conductivity. The flow meter, pH and conductivity allow for real-time accurate determination of inlet water conditions. Hardness, Ca ion-like ions and alkalinity are analytical instruments, preferably online instruments that can be monitored in real time, more preferably with monitoring statistics data at a frequency of once every 2 h. Ca ion-like ions refer to ions that cause water hardness to rise, such as Ca²⁺, Mg²⁺.

The inlet water monitoring data is input to the intelligent dosing control device in real time, and the accurate dosing value is obtained after accurate calculation according to the inlet water quality and expected outlet water quality (outlet water quality requirements) by the intelligent dosing control device. The calculated value is then subject to fuzzy data adjustment, comparison with the memory data in the database to output the fuzzy factor coefficient f for correcting the agent dosing value.

The correction results are output to the agent dosing device. By accurate control of the dosage of the agent dosing device, the agent is dosed into the hardness removal device. The agent dosing device may be a dosing pump or other dosing device that is capable of dosing.

The hardness removal device may be a high-density sedimentation tank or other devices that allow for removing hardness by agents.

Monitoring instruments are arranged at the outlet pipe of the hardness removal device for monitoring hardness, Ca ion-like ions, alkalinity, pH and conductivity. The monitoring data is fed back to the intelligent dosing control device in real time. If the produced water quality meets the expectations (requirements), it will become a multi-dimensional data point under this condition, which will be memorized and stored in the intelligent dosing system database. If there is a deviation from the expectation, the data will be adjusted and accounted by the intelligent dosing control device before being output to the agent dosing device.

After running for a period of time, the intelligent dosing control device can accumulate abundant data of working condition control parameters. Such data is used to correct the theoretically accurate calculated value to obtain the fuzzy factor coefficient f, thus realizing the quantitative control of the output fuzzy influencing factors on the agent dosage.

The present disclosure will be described in detail with reference to examples below.

### EXAMPLES AND COMPARATIVE EXAMPLES

A zero-discharge project in a coal chemical industry in Baotou was designed with a treatment capacity of 400 t/h, wherein the treatment process was an integrated process route of “pre-treatment-membrane treatment-silicon removal treatment-ozone catalytic oxidation-nanofiltration salt separation-quality grading crystallization”. A high-density sedimentation tank was used in the pre-treatment stage to remove the hardness and alkalinity in water to ensure the stable operation of the subsequent membrane treatment.

The conditions of actual inlet and outlet water and agent dosing in the high-density sedimentation tank in the pre-treatment stage were monitored by monitoring instruments for flow rate, hardness, Ca²⁺, Mg²⁺, alkalinity, pH, conductivity, and the like, wherein the flow rate, pH and conductivity were data statistics from real-time monitoring in field, and the hardness, calcium ion, magnesium ion, and alkalinity were data statistics that were measured through the field sampling and analysis by the laboratory every 12 hours. The dosed agents were a sodium hydroxide solution at a concentration of 32% and a sodium carbonate solution at a concentration of 10%. As the water treatment time increased, each specific dosing and statistical results were shown in Table 1 below.

TABLE 1
inlet and outlet water and agent dosing in the high-density sedimentation tank

| Name | Item | Unit | 1# | 2# | 3# | 4# | 5# | 6# | 7# | 8# | 9# | 10# |
|---|---|---|---|---|---|---|---|---|---|---|---|---|
| Inlet | Flow rate | m³/h | 217.44 | 236.55 | 334.44 | 333.31 | 308.86 | 307.78 | 322.67 | 332.68 | 305.82 | 316.08 |
| water of | | | | | | | | | | | | |
| high- | | | | | | | | | | | | |
| density | | | | | | | | | | | | |
| sedi- | | | | | | | | | | | | |
| menta- | | | | | | | | | | | | |
| tion | | | | | | | | | | | | |
| tank | | | | | | | | | | | | |
| Inlet | pH | / | 7.84 | 8.21 | 8.69 | 8.48 | 8.41 | 8.61 | 8.79 | 8.58 | 8.07 | 8.85 |
| water | Conduc- | us/cm | 16980 | 15780 | 15970 | 14790 | 14790 | 16400 | 14820 | 13280 | 15920 | 17020 |
| quality | tivity | | | | | | | | | | | |
| of high- | Total | mg/L | 1960 | 1580 | 1350 | 1508 | 1524 | 1200 | 928 | 1090 | 1012 | 800 |
| density | hardness | | | | | | | | | | | |
| sedi- | (as | | | | | | | | | | | |
| menta- | calcium | | | | | | | | | | | |
| tion | carbonate) | | | | | | | | | | | |
| tank | Calcium | mg/L | 1400 | 1125 | 1012.5 | 1012.5 | 1012.5 | 900 | 675 | 675 | 675 | 675 |
| | ion (as | | | | | | | | | | | |
| | calcium | | | | | | | | | | | |
| | carbonate) | | | | | | | | | | | |
| | Magnesium | mg/L | 560 | 455 | 337.5 | 495.5 | 511.5 | 300 | 253 | 415 | 337 | 125 |
| | ion (as | | | | | | | | | | | |
| | calcium | | | | | | | | | | | |
| | carbonate) | | | | | | | | | | | |
| | Alkalinity | mg/L | 400 | 500 | 800 | 750 | 700 | 550 | 900 | 650 | 650 | 900 |
| | (as | | | | | | | | | | | |
| | calcium | | | | | | | | | | | |
| | carbonate) | | | | | | | | | | | |
| Outlet | pH | / | 10.94 | 11.01 | 10.77 | 10.91 | 10.76 | 10.69 | 10.46 | 11.02 | 11.05 | 10.77 |
| water | Total | mg/L | 28 | 32 | 38 | 45 | 28 | 67.5 | 30 | 24 | 90 | 42 |
| quality | hardness | | | | | | | | | | | |
| of high- | (as | | | | | | | | | | | |
| density | calcium | | | | | | | | | | | |
| sedi- | carbonate) | | | | | | | | | | | |
| menta- | Calcium | mg/L | 22.5 | 22.5 | 22.5 | 42 | 11.2 | 50 | 22.5 | 22.5 | 67.5 | 22.5 |
| tion | ion (as | | | | | | | | | | | |
| tank | calcium | | | | | | | | | | | |
| | carbonate) | | | | | | | | | | | |
| | Magnesium | mg/L | 5.5 | 9.5 | 15.5 | 3 | 16.8 | 12.5 | 7.5 | 1.5 | 22.5 | 19.5 |
| | ion (as | | | | | | | | | | | |
| | calcium | | | | | | | | | | | |
| | carbonate) | | | | | | | | | | | |
| | Alkalinity | mg/L | 1550 | 1850 | 2050 | 2300 | 2000 | 1500 | 1700 | 1500 | 1400 | 1850 |
| | (as | | | | | | | | | | | |
| | calcium | | | | | | | | | | | |
| | carbonate) | | | | | | | | | | | |
| Manual | 32% sodium | L/H | 420 | 630 | 533 | 643 | 653 | 943 | 440 | 570 | 600 | 560 |
| con- | hydroxide | | | | | | | | | | | |
| trolled | dosage | | | | | | | | | | | |
| agent | 10% sodium | L/H | 1910 | 3170 | 2670 | 3110 | 3140 | 2680 | 2620 | 2360 | 2670 | 2620 |
| dosage | carbonate | | | | | | | | | | | |
| | dosage | | | | | | | | | | | |
| The | 32% sodium | L/H | 355 | 384 | 656 | 720 | 648 | 440 | 642 | 608 | 515 | 554 |
| intel- | hydroxide | | | | | | | | | | | |
| ligent | dosage | | | | | | | | | | | |
| dosing | 10% sodium | L/H | 1498 | 439 | 0 | 0 | 0 | 0 | 0 | 0 | 0 | 0 |
| system- | carbonate | | | | | | | | | | | |
| recom- | dosage | | | | | | | | | | | |
| mended | | | | | | | | | | | | |
| agent | | | | | | | | | | | | |
| dosage | | | | | | | | | | | | |

Table 1 indicated that the hardness and alkalinity of the inlet water are high and fluctuate greatly, with a maximum and minimum total hardness of 1960 mg/L and 800 mg/L, respectively, and a maximum and minimum alkalinity of 900 mg/L and 400 mg/L, respectively, which belongs to the situation where the 2× carbonate alkalinity of raw water is less than the calcium hardness of raw water.

The total hardness can be reduced to less than 50 mg/L by manual control of dosing of sodium hydroxide and sodium carbonate (Model 6) softening agents in field. But the alkalinity of the produced water reached 1400 mg/L, even 2300 mg/L. According to the analysis of the hardness, calcium ion, magnesium ion, alkalinity of the inlet water, and the hardness, calcium ion, magnesium ion, alkalinity of the outlet water, the effect of removing hardness of the produced water basically meets the expectations, but the produced water was enriched with large quantities of alkalinity.

By using the intelligent dosing control method for softening and removing hardness of the disclosure, agent dosage calculation and recommendation were carried out based on real-time inlet and outlet water conditions in the above treatment process in field. The agent scheme recommended that model 4 (sodium hydroxide) was used for later stage and model 6 (sodium hydroxide combined with sodium carbonate) was used at the early stage, see table 1 for the specific recommended dosages. The first level of accurate calculation process involved was shown in table 2 below:

TABLE 2

| I | Condition input | Value | Calculation | note |
|---|---|---|---|---|
| 1 | Flow rate | Q(m³/h) | | Q is from real-time instrument |
| | | | | data in field |
| 2 | Sodium | C_{NaOH} | C_{NaOH} = a₁*a₂ | Sodium hydroxide purity a₁ * |
| | hydroxide | | | dispensing concentration a₂ |
| 3 | Sodium | C_{Na2CO3} | C_{Na2CO3} = b₁*b₂ | Sodium carbonate purity b₁ * |
| | carbonate | | | dispensing concentration b₂ |
| II | Water | Volume | Molar | Input values is in the unit of |
| | quality | concentration | Concentration | CaCO₃ |
| | input | (mg/L) | (mL/L) | |
| 1 | Inlet water | X1 | A1 = X1/50 | X1 is from data measured by a |
| | Ca²⁺ | | | calcium hardness meter in field |
| 2 | Inlet water | X | B1 = (X − X1)/50 | X is from data measured by a |
| | Mg ²⁺ | | | total hardness meter in field |
| 3 | Inlet | Z1 | C1 = Z1/50 | Z1 is from data measured by an |
| | water | | | alkalinity meter in field |
| | alkalinity | | | |
| 4 | Outlet | X2 | A2 = X2/50 | The data is from the outlet water |
| | water | | | quality required for adjusting; |
| | Ca²⁺ | | | X2 = 50 mg/L, general |
| | | | | requirements: 50-100 mg/L; |
| | | | | When X2 > X1 is entered, an |
| | | | | input error is prompted; |
| 5 | Outlet water | Y | B2 = Y/50 | The data is from the outlet water |
| | Mg²⁺ | | | quality required for adjusting; |
| | | | | Y = 25 mg/L, general |
| | | | | requirements: 25-50 mg/L; |
| | | | | When B2 > B1 is entered, an |
| | | | | input error is prompted; |
| 6 | Outlet water | Z2 | C2 = Z2/50 | The data is from the outlet water |
| | alkalinity | | | quality required for adjusting; |
| | | | | Z2 = 50 mg/L, general |
| | | | | requirements: 25-50 mg/L; |
| | | | | When Z2 > Z1 is entered, an input |
| | | | | error is prompted; |
| III | Model | Calculation and judgment process | | A = A1 − A2, B = B1 − B2, |
| | selection | | | C = C1 − C2, carbon dioxide and |
| | | | | flocculant could not be measured |
| | | | | due to a tiny amount |
| 1 | Model 4 | A ≤ 2*C | | Ca²⁺ equivalent is not greater |
| | | | | than 2x HCO₃⁻ equivalent |
| 2 | Model 6 | A > 2*C | | Ca²⁺equivalent is greater |
| | | | | than 2x HCO₃⁻ equivalent |
| IV | Output | Item | Calculation process | Dosing results (see Table 1 for |
| | | | | details) |
| 1 | Model 4 | NaOH | =Q(B + C)*40/1000/C_{NaOH} | Sodium hydroxide dosing pump |
| | | | | (L/H) |
| | | Na₂CO₃ | =0 | Sodium carbonate does not need |
| | | | | to be added under current water |
| | | | | quality conditions |
| 2 | Model 6 | NaOH | =Q (B + C) *40/1000/C_{NaOH} | Sodium hydroxide dosing pump |
| | | | | (L/H) |
| | | Na₂CO₃ | =Q* (A − 2*C) *53/1000/C_{Na2CO3} | Sodium carbonate dosing pump |
| | | | | (L/H) |

By comparing the field manual controlled actual dosage (a comparative example) with the intelligent dosing system recommended dosage (an example) in table 1, it can be seen that, for sodium hydroxide, the dosing flow rate value of the comparative example is basically the same as the recommended dosage of the example, but for sodium carbonate, the recommended flow rate values of the example were 1498 L/h and 439 L/h at beginning, and zero was recommended for a later time, while the dosing operation was maintained throughout the process in field in the comparative example, resulting in a substantial overdose, which also accounts for large quantities of alkalinity occurring in the outlet water.

Therefore, the agent dosage can be recommended in real time by the intelligent dosing control method for softening and removing hardness based on the changes in the inlet water quality and quantity, to adjust the dosing of sodium hydroxide and sodium carbonate, thereby avoiding the overdosage and saving a lot of agent costs. Based on overdosing 10% sodium carbonate by 2500 L/h in field, and assuming a sodium carbonate price of 3000 yuan/ton, it can be estimated that the annual savings in sodium carbonate costs will be over 6.4 million yuan, and the cost per ton of water will be reduced by 1.875 yuan/ton. The outlet water alkalinity level from the high-density sedimentation tank has been reduced, which also saved the subsequent acid consumption for removal of excess alkalinity. This will save the annual cost of acid reagent more than 3.5 million yuan, and reduce the cost of per ton of water by 1.03 yuan/t. As a result of reducing excessive introduction of salt, the salt content entering the subsequent sections of membrane concentration and evaporation and crystallization will be reduced, leading to a decrease in the operating pressure in the sages of membrane concentration and evaporation and crystallization, thereby realizing a more stable operation.

The above is only a preferred example of the present disclosure, and is not intended to limit the present disclosure, any modification, equivalent substitution, improvement, and the like made within the spirit and principle of the disclosure should be included within the scope of the claims of the disclosure.

What is claimed is:

1. An intelligent dosing control method for softening and removing hardness, comprising:
(1) recommending an agent scheme, including: analyzing and comparing a plurality of agent dosing schemes based on different inlet water qualities and produced water quality requirements, and then recommending an appropriate agent dosing scheme based on agent cost and sludge production;
(2) precisely controlling the agent dosing, including:
a first level: accurate data calculation, wherein a theoretical agent dosage is obtained by calculating accurately and analyzing inlet water flow rate and calcium ion, magnesium ion, and alkalinity in the inlet and outlet water, yₘ represents theoretical agent dosage obtained after m-th accurate calculation and analysis, where m is a positive integer greater than or equal to 1;
a second level: fuzzy data adjustment, comprising a first stage of memory and analysis and a second stage of output and readjustment,
in the first stage of memory and analysis: dosing by taking yₘ (m≤n) obtained from accurate data calculation of the first level as a basis for expected agent dosing, then performing analysis and adjustment of the agent dosage and a secondary dosing according to feedback of the produced water quality, and recycling the analysis and adjustment of agent dosage based on the feedback of produced water quality until the produced water quality meets a requirement, stopping recycle, performing statistics of actual agent consumption to obtain an actual agent dosage y; then conducting fuzzy factor function analysis y=f(yₘ, X) based on y and yₘ to give the fuzzy factor function f(yₘ, X), where X represents influencing factors that have no determined impact on outlet water quality, the fuzzy factor function f(yₘ, X) is then combined with actual inlet and outlet water quality as well as the agent dosing scheme and agent dosing parameters yₘ and y to form a multi-dimensional data point, which is stored in memory; upon a memory database is formed by accumulating n multi-dimensional data points, entering the second stage of output and readjustment, where n is a positive integer greater than or equal to 1000;
in the second stage of output and readjustment: simultaneously introducing the inlet water and produced water quality requirements and yₘ (m>n) obtained according to accurate data calculation of the first level into the fuzzy data adjustment of the second level, and performing comparison and analysis with the multi-dimensional data points in the memory database to give the corresponding fuzzy factor function f(yₘ, X), and outputting an agent dosing parameter including y corresponding to f(yₘ, X), then performing a predictable agent dosing according to the parameter using y as predictable dosing basis; after receiving the feedback of actual produced water quality, performing comparison and analysis with the produced water quality requirements: if there is no difference, determining that predictable agent parameter adjustment and dosing are achievable; if there is a difference, performing readjustment of the agent dosing according to the actual produced water quality and requirements on produced water quality, i.e., recycling the analysis and adjustment of agent dosage based on the feedback of produced water quality until the actual produced water quality meets the requirement on the produced water quality, stopping the recycle, performing statistics of the actual agent consumption to obtain an actual agent dosage z; then conducting fuzzy factor function analysis z=f(y, X) based on y and z to give the fuzzy factor function f(y, X), thereby realizing the readjustment of the fuzzy factor function f(yₘ, X), while forming a new multi-dimensional data point including the actual inlet and outlet water quality, the agent dosing scheme, y, z, and f(y, X), which is stored in memory; thereby realizing a intelligent agent dosing control through continuous output adjustment and memory.

2. The intelligent dosing control method for softening and removing hardness according to claim 1, wherein there are at least two of the agent dosing schemes.

3. The intelligent dosing control method for softening and removing hardness according to claim 2, wherein there are at most seven of the agent dosing schemes.

4. The intelligent dosing control method for softening and removing hardness according to claim 1, wherein the agent dosing scheme includes two or more of model 1: calcium hydroxide; model 2: calcium hydroxide+sodium carbonate; model 3: calcium hydroxide+sodium carbonate+trisodium phosphate; model 4: sodium hydroxide; model 5: calcium hydroxide+sodium hydroxide; model 6: sodium hydroxide+sodium carbonate; model 7: lime+gypsum or lime+calcium chloride.

5. The intelligent dosing control method for softening and removing hardness according to claim 4, wherein the model 1 is suitable for wastewater with low hardness and high alkalinity; and/or
the model 2 is suitable for wastewater with high hardness and low alkalinity; and/or
the model 3 is suitable for wastewater which is treated with lime+soda ash to give a residual hardness of 0.15-0.2 mmol/L, and needs further softening to obtain a residual hardness of 10-20 μmol/L; and/or
the model 4 is suitable for wastewater where 2× carbonate alkalinity of raw water is not less than calcium hardness of raw water; preferably, the model 4 is suitable for wastewater where the 2× carbonate alkalinity of raw water is equal to the calcium hardness of raw water; the model 5 is suitable for wastewater where the 2× carbonate alkalinity of raw water is more than the calcium hardness of raw water; and/or
the model 6 is suitable for wastewater where the 2× carbonate alkalinity of raw water is less than the calcium hardness of raw water; and/or
the model 7 is suitable for negative hard water having a total alkalinity of raw water greater than or equal to a total hardness of raw water of 2 mmo/L.

6. The intelligent dosing control method for softening and removing hardness according to claim 1, wherein the influencing factor X includes one or more of accuracy of various measuring instruments, a fluid discharge condition of a dosing device, a operating temperature and a reaction time.

7. An intelligent dosing control device for softening and removing hardness, employing the intelligent dosing control method for softening and removing hardness according to claim 1.

8. An intelligent dosing control system for softening and removing hardness, comprising the intelligent dosing control device for softening and removing hardness according to claim 7.

9. The intelligent dosing control system for softening and removing hardness according to claim 8, further comprising: monitoring instruments, arranged at an inlet pipe of the softening and hardness removal process section, for monitoring flow rate, hardness, Ca ion-like ions, alkalinity, pH and conductivity, to transmit inlet water monitoring data to the intelligent dosing control device for softening and removing hardness; and
an agent dosing device and a hardness removal device, wherein the intelligent dosing control device for softening and removing hardness outputs an agent dosage to the agent dosing device, to enable the agent to be accurately dosed into the hardness removal device for softening and removing hardness of wastewater; and
monitoring instruments, arranged at an outlet pipe of the hardness removal device, for monitoring flow rate, hardness, Ca ion-like ions, alkalinity, pH and conductivity, to transmit outlet water monitoring data to the intelligent dosing control device for softening and removing hardness.

10. The intelligent dosing control system for softening and removing hardness according to claim 9, wherein the monitoring instruments are online real-time monitoring instruments that measure inlet and/or outlet water in real time; preferably, frequency of monitoring statistics data is once every 2 hours.

11. The intelligent dosing control method for softening and removing hardness according to claim 2, wherein the agent dosing scheme includes two or more of model 1: calcium hydroxide; model 2: calcium hydroxide+sodium carbonate; model 3: calcium hydroxide+sodium carbonate+trisodium phosphate; model 4: sodium hydroxide; model 5: calcium hydroxide+sodium hydroxide; model 6: sodium hydroxide+sodium carbonate; model 7: lime+gypsum or lime+calcium chloride.

12. The intelligent dosing control method for softening and removing hardness according to claim 3, wherein the agent dosing scheme includes two or more of model 1: calcium hydroxide; model 2: calcium hydroxide+sodium carbonate; model 3: calcium hydroxide+sodium carbonate+trisodium phosphate; model 4: sodium hydroxide; model 5: calcium hydroxide+sodium hydroxide; model 6: sodium hydroxide+sodium carbonate; model 7: lime+gypsum or lime+calcium chloride.

13. An intelligent dosing control device for softening and removing hardness, employing the intelligent dosing control method for softening and removing hardness according to claim 2.

14. An intelligent dosing control device for softening and removing hardness, employing the intelligent dosing control method for softening and removing hardness according to claim 3.

15. An intelligent dosing control device for softening and removing hardness, employing the intelligent dosing control method for softening and removing hardness according to claim 4.

16. An intelligent dosing control device for softening and removing hardness, employing the intelligent dosing control method for softening and removing hardness according to claim 5.

17. An intelligent dosing control device for softening and removing hardness, employing the intelligent dosing control method for softening and removing hardness according to claim 6.
